(19) United States
(12) Patent Application Publication
(10) Pub. No.: US 2015/0238441 A1
(43) Pub. Date: Aug. 27, 2015

(54) Modulation of Branched Amino Acid Concentrations to Treat Metabolic Diseases

(71) Applicant: UNIVERSITY OF ROCHESTER, Rochester, NY (US)

(72) Inventor: David S. Goldfarb, Victor, NY (US)

(73) Assignee: University of Rochester, Rochester, NY (US)

(21) Appl. No.: 14/433,429

(22) PCT Filed: Oct. 7, 2013
(86) PCT No.: PCT/US13/63669

§ 371 Date: Apr. 3, 2015

Related U.S. Application Data
(60) Provisional application No. 61/710,218, filed on Oct. 5, 2012.

(51) Int. Cl. A61K 31/122; A61K 45/06

(52) U.S. Cl. CPC A61K 31/122; A61K 45/06

(57) ABSTRACT

The plasma concentration of at least one branched chain amino acid in a mammal in need of such reduction is reduced by administering an agent that increases the plasma concentration of large neutral amino acids. The invention also includes related compositions and methods. These methods and compositions can be used to treat insulin resistance, type 2 diabetes and metabolic syndrome.

### CROSS-REFERENCE TO RELATED APPLICATIONS

This application claims priority to U.S. Provisional Application No. 61/710,218, filed on Oct. 5, 2012, now pending, the disclosure of which is incorporated herein by reference.

### BACKGROUND

Worldwide increases in the incidence of type 2 diabetes are largely the result of poor diet, obesity, and sedentary lifestyle. An estimated 171 million people worldwide were diagnosed as having type 2 diabetes in 2000 and that number is projected to increase to 366 million by 2030, resulting in high morbidity and increased economic burden.

The pathophysiology of type 2 diabetes is characterized by decreased insulin sensitivity, deterioration of pancreatic islet cell function and decreased incretin function. The disease is progressive and the resulting loss of insulin function leads to chronic hyperglycemia and is associated with severe vascular complications caused by excessive protein glycation and oxidative stress. The goal of current therapeutics is to reduce all of the components of dysglycemia. While several classes of oral anti-diabetic drugs have been approved, their success is limited by their mechanisms of action, which often target the symptoms of diabetes rather than the underlying pathophysiology. Another limitation of current drugs is that they are all associated with significant side effects. Approximately 20% of patients taking sulfonylurea drugs such as Glucotrol, which acts to increase insulin release, experience significant hypoglycemia. More than 60% of patients taking metformin, which suppresses hepatic glucose production, suffer gastrointestinal side effects. Patients taking thiazolidinediones such as Actos® and Avandia®, which increase insulin sensitivity, often suffer peripheral edema. Actos® was recently removed from the market in Europe due to cardiovascular risk. Most of these therapies are associated with significant weight gain. Clearly, new classes of therapeutics that target core metabolic pathways that underlie the basis of disease pathology are needed.

Obesity-related maladies (i.e. “metabolic diseases”) are due, in part, to the impact of excess lipids on various cellular functions. In addition to lipids, certain amino acids may be both markers and effectors of insulin resistance (reviewed in Newgard, 2012). It has been known for decades that high fasting serum concentrations of branched chain amino acids (BCAAs) and aromatic amino acids (AAA) were correlated with obesity and serum insulin (Felig et al., 1969). Importantly, the probability that normoglycemic individuals will develop insulin resistance was most strongly correlated with fasting levels of three BCAAs (leucine, isoleucine, valine) and two AAAs (phenylalanine and tyrosine) (Wang et al., 2011). Rats fed extra BCAAs displayed a greater tendency to develop obesity-associated insulin resistance (Newgard et al., 2009). This study concluded that BCAAs contribute to the development of insulin resistance, a prerequisite for the development of type II diabetes. A report that leucine deprivation increased hepatic insulin sensitivity supports the hypothesis that BCAAs have a causative role in insulin resistance (Xiao et al., 2011).

A rationale for the association of high BCAA levels with metabolic diseases is based on their known metabolic interactions with lipids (Newgard, 2009, Newgard, 2012). The association of AAAs with risk of insulin resistance is less well understood; however, elevated plasma levels of AAAs are possibly an indirect consequence of elevated BCAAs. BCAAs and large neutral amino acids (LNAAs) compete for the same plasma membrane transporters (Verrey, 2003). High BCAA levels may saturate these transporters and lead to the incidental accumulation of other LNAAs such as the aromatic amino acids phenylalanine, tyrosine and tryptophan. This theory found practical application in the treatment of phenylketonuria (PKU), which is commonly treated by reducing dietary phenylalanine. In rats, a diet supplemented with LNAAs lacking phenylalanine reduced brain phenylalanine concentrations (reviewed van Spronsen et al., 2010). Moreover, PKU patients supplemented with valine, isoleucine and leucine exhibited improved neuropsychological functions (see van Spronsen et al., 2010). In summary, there is experimental support for the hypothesis that levels of specific LNAAs can be modulated by high dietary doses of other LNAAs. One limitation to this approach is a difficulty in maintaining high plasma levels of competing amino acids, which may rise following consumption, but drop in the hours after the meal and especially during overnight fasts (van Spronsen et al. 2001).

A strict inverse relationship between plasma BCAA and LNAA levels is not supported by measurement of basal plasma amino acid levels in PKU patients. In one study, it was determined that these patients had phenylalanine levels that were 15-30 times higher than controls, but their plasma BCAA levels were no different than controls (Pietz et al. 1999). In contrast, a separate study showed that plasma levels of BCAA and other amino acids are lower in PKU patients with elevated phenylalanine levels compared to healthy patients (Efron, et al. 1969). In still another study, patients with Maple Syrup Urine Disease (MSUD), who have decreased activity of the branched-chain alpha-ketoacid dehydrogenase complex (BCKAD), the second enzymatic step in the degradative pathway of the BCAAs demonstrated dramatically elevated plasma levels of BCAAs, with leucine levels being approximately 30 fold higher than controls. However, patients with MSUD either present with plasma LNAA concentrations comparable to non-affected individuals, or with lower plasma levels of all groups of amino acids including LNAA and charged amino acids (Strauss, et al. 2010). In summary, the physiological consequences of modulating plasma amino acid levels by diet cannot be predicted based upon the changes in plasma levels of amino acids that result from metabolic disorders, such as occurs in PKU and MSUD. Differences between amino acid metabolism within different tissues and organs, and the interplay between amino acid metabolism and other pathways, such as lipid oxidation and the TCA cycle, make it difficult to predict the outcome of therapeutic interventions that target amino acid metabolism.

Obesity in mice and humans is associated with higher levels of BCAA and acylcarnitines associated with the catabolism of BCAA, as well as lipid derived metabolites released from adipose tissue (Muoio et al., 2008). It is generally acknowledged that the synergy of elevated BCAA and the accumulation of free fatty acids in the plasma of obese individuals mediate many adverse metabolic effects including insulin resistance and increased risk for cardiovascular disease. (Muoio et al., 2008).

Thus, the invention provides methods and compositions designed to decrease BCAA levels and levels of deleterious BCAA metabolites, such as acylcarnitine-derivatives, other molecules produced by incomplete oxidation of BCAA, and free fatty acids. As such, the invention provides methods and compositions that are useful in treating obesity, diabetes and metabolic disease.

### SUMMARY OF THE INVENTION

In one aspect, the invention provides a method of reducing the plasma concentration of at least one branched chain amino acid in a mammal in need of such reduction that includes the step of administering to the mammal an agent that increases the plasma concentration of at least one, but not all, large neutral amino acids. In certain embodiments, the agent is not a leucine supplement. In certain other embodiments, the agent is not a dietary or nutraceutical source of one or more amino acids.

In another aspect, the invention provides a method of reducing the plasma concentration of at least one free fatty acid and/or at least one fatty acid oxidation metabolite, and/or at least one metabolite of triglyceride oxidation, in a mammal in need of such reduction that includes the step of administering to the mammal an agent that increases the plasma concentration of at least one, but not all, large neutral amino acids. In certain embodiments, the agent is not a leucine supplement. In certain other embodiments, the agent is not a dietary or nutraceutical source of one or more amino acids.

In another aspect, the invention provides a pharmaceutical composition comprising:
a. an HPPD inhibitor;
b. a second therapeutic agent selected from an agent useful to treat insulin resistance, type 2 diabetes, metabolic syndrome, or obesity; or a dietary tyrosine supplement; and
c. a pharmaceutically acceptable carrier.

The invention also includes the use of the compounds described herein for the treatment of one or more of the conditions described herein. In addition, the invention includes the use of compounds described herein for the manufacture of medicaments for treating one or more of the conditions described herein.

### BRIEF DESCRIPTION OF THE DRAWINGS

FIG. 1 depicts how inhibition of HPPD causes the depletion of downstream toxic metabolites such as succinlyacetoacetate in type 1 tyrosinemia patients. These patients are deficient in fumarylacetoacetate hydroxylase (FAH). In contrast, the upstream metabolites 4-hydroxyphenylpyruvate (4-HPP) and tyrosine accumulate and may be excreted. Figure taken from Lock et al., 1998.

FIG. 2A depicts how a single 1 mg/kg oral gavage dose of NTBC (Compound 2) affected plasma amino acid levels at six and twelve hours in mice. FIG. 2B depicts the relative level of tyrosine and tyrosine metabolites six and twelve hours after administration as compared to vehicle alone. In FIGS. 2A and 2B, a cell shaded green indicates a significant difference from the starting value (p≦0.05) and a ratio of <1 relative to the starting value, while a cell shaded red indicates a significant difference from the starting value (p≦0.05) and a ratio of >1 relative to the starting value. Unshaded cells are not significantly different from the starting values.

FIG. 3 depicts the effect of a single 1 mg/kg oral gavage dose of NTBC on plasma levels of isovalerylcarnitine in mice as compared to vehicle alone after six and 12 hours.

FIG. 4 depicts the effect of a single 1 mg/kg oral gavage dose of NTBC on plasma levels of gamma glutamyl amino acid in mice as compared to vehicle alone after six and 12 hours. In FIG. 4, a cell shaded green indicates a significant difference from the starting value (p≦0.05) and a ratio of <1 relative to the starting value, while a cell shaded red indicates a significant difference from the starting value (p≦0.05) and a ratio of >1 relative to the starting value. Numbers bolded in blue represent a statistical difference from starting value with a p value of greater than 0.05 and less than 0.10.

FIG. 5 depicts the effect of a single 1 mg/kg oral gavage dose of NTBC on plasma levels of catabolic intermediates of isoleucine (2-hydroxy-3-methylvalerate) and leucine (α-hydroxyisocaproate) in mice as compared to vehicle alone after six and 12 hours.

FIG. 6 depicts the effect of a single 1 mg/kg oral gavage dose of NTBC on the plasma levels of various monoacylglycerides (Panel A) and free fatty acids (Panel B) in mice as compared to vehicle alone after six and 12 hours.

### DETAILED DESCRIPTION OF THE INVENTION

### General Description of Certain Aspects of the Invention

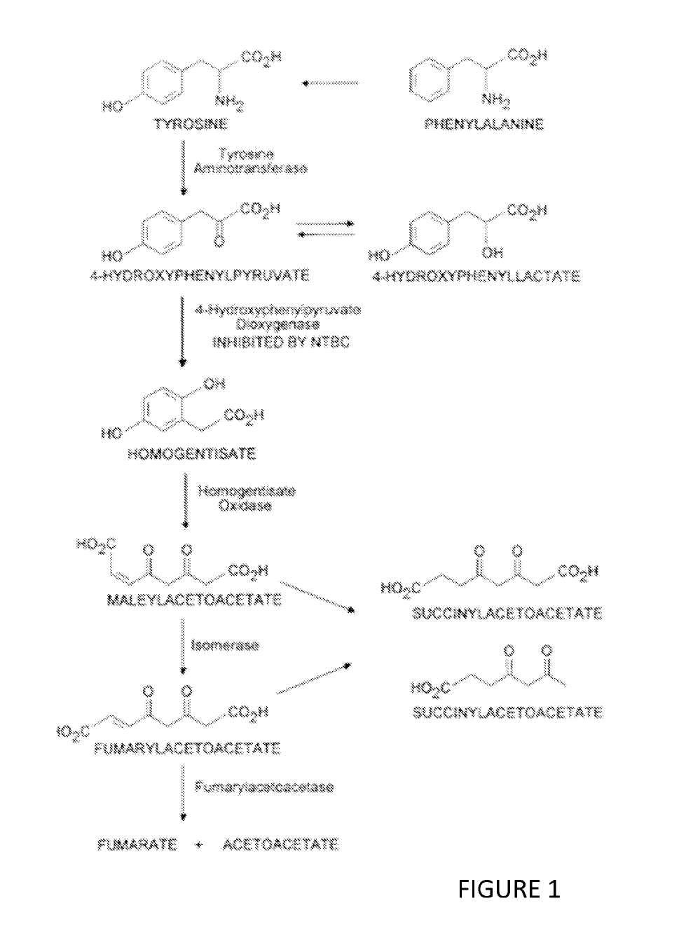

We propose a novel method to reduce plasma levels of BCAAs that is more efficient and long lasting than dietary supplements of LNAAs. Nitisinone (also known as NTBC) was originally developed as an herbicide (Lock et al., 1998; Beaudegnies, R. et al., 2009), and acts by inhibiting hydroxyphenylpyruvate dioxygenase (HPPD), an enzyme in tyrosine catabolism that is conserved in mammals (FIG. 1). In vivo inhibition of HPPD in rodents and humans causes the accumulation of plasma tyrosine (Lock et al., 2000). NTBC is currently administered to patients with adolescent type I tyrosinemia, who would otherwise accumulate toxic metabolites and die (Lock et al., 1998; see FIG. 1).

NTBC is an irreversible inhibitor of HPPD and as such effectively elevates tyrosine levels and, moreover, maintains consistently high plasma tyrosine levels for more than one full day. A single 10 μM (micromolar)/kg oral dose of NTBC increased plasma tyrosine levels two-fold within 30 min and over seven-fold at 16 h (Locke et al. 2000). Levels remained elevated more than two-fold three days after dosing. In an earlier study, a 0.2 mg/kg NTBC dose elevated tyrosine levels nine-fold at 24 hrs (Lock et al. 1996). Doses of NTBC significantly less than prescribed for adolescent type 1 tyrosinemia may suffice for the purpose of reducing plasma BCAA levels. Combinations of tyrosine and NTBC or other LNAAs and NTBC may be more effective than NTBC itself at lower doses.

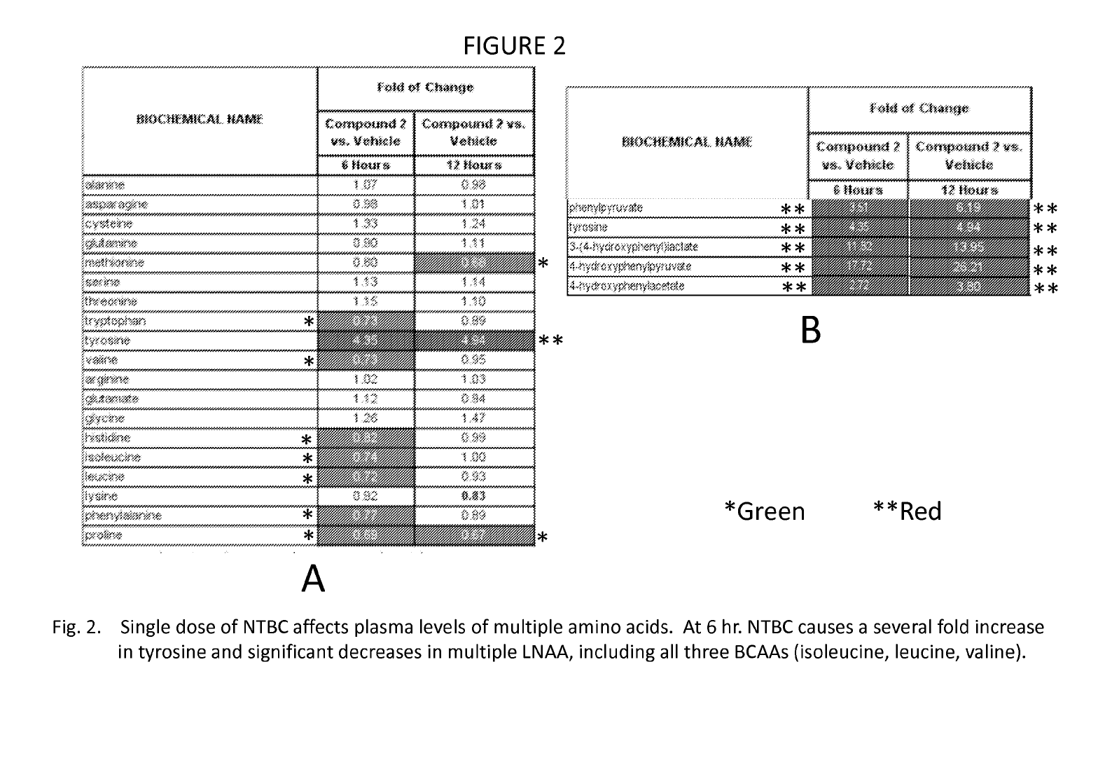

It has now been found that an agent that increases levels of tyrosine, one of the LNAAs, causes a decrease in plasma BCAAs. For example, six male C57BL/6 mice (20-25 gm, 8-9 weeks age) were administered a single aqueous 1 mg/kg dose of NTBC by oral gavage. Plasma samples were taken 6 h following dosing. As shown in FIG. 2, a single dose of NTBC resulted in elevated plasma levels of tyrosine and reduced levels of valine, isoleucine and leucine (BCAAs) by greater than 25%. We conclude that NTBC can be used to reduce plasma BCAA levels in mice. It should be noted that an early study reported that plasma BCAA levels were unaffected after dosing rats with 10 mg/kg NTBC for 11 weeks (Lock et al., 1996).

In addition, it has been found that an agent that increases levels of tyrosine also causes a statistically significant decrease in at least one free fatty acid and/or at least one fatty acid oxidation metabolite, and/or at least one metabolite of triglyceride oxidation, such as monoacylglycerides and free fatty acids. Such metabolites are known to be increased in obesity, diabetes.

In certain embodiments, an agent that increases the concentration of at least one LNAA, but not all LNAAs, is used in a method of reducing the concentration of BCAAs in a mammal that needs such reduction. As used herein, the terms “BCAA” and “branched chain amino acid” are used interchangeably and refer to any of leucine, isoleucine or valine. As used herein, “LNAA” and “large neutral amino acid” are used interchangeably and refer to any of leucine, isoleucine, valine, methionine, tyrosine, phenylalanine, histidine or tryptophan. In certain embodiments, the method reduces the concentration of one, two or all three BCAAs.

For the methods of the invention, the concentrations of one or more BCAAs, or at least one free fatty acid and/or at least one fatty acid oxidation metabolite, and/or at least one metabolite of triglyceride oxidation are reduced, for example, in the plasma and/or intracellularly in one or more tissues. In certain embodiments, the plasma concentration of BCAAs serves as a marker of intracellular concentration of BCAAs. In certain embodiments, the plasma concentration of at least one free fatty acid and/or at least one fatty acid oxidation metabolite, and/or at least one metabolite of triglyceride oxidation serves as a marker of intracellular concentration of that free fatty acid, fatty acid oxidation metabolite, or metabolite of triglyceride oxidation. It should be understood that the terms “at least one” and “one or more” as used herein are intended to be interchangeable and both mean in various embodiments of the invention 1, 2, 3, 4, 5, 6, 7, 8, 9, 10, 11, 12, or more.

Agents that are capable of increasing the concentration of one or more LNAA (“LNAA-increasing agents”), may cause increased concentration in plasma or intracellularly. LNAA-increasing agents include agents that increase the concentration of one or more of tyrosine, phenylalanine, tryptophan, methionine and histidine, particularly tyrosine. In certain embodiments, the LNAA-increasing agent is not leucine. In certain embodiments, the LNAA-increasing agent is not a naturally-occurring amino acid. In certain embodiments, the LNAA-increasing agent is not an ingestible source of one or more amino acids, e.g., not a dietary source of one or more amino acids, e.g., not an ingestible amino acid supplement, peptide, polypeptide, or protein. In certain embodiments, the LNAA-increasing agent is a HPPD inhibitor, such as nitisinone.

Advantageously, an LNAA-increasing agent used herein increases the concentration of one or more LNAAs for at least 6 hours. In some aspects, an LNAA-increasing agent used herein increases the concentration of one or more LNAAs for at least 6, 7, 8, 9, 10, 11, or 12 hours. In one aspect, an LNAA-increasing agent used herein increases the concentration of one or more LNAAs for no more than 12 hours.

LNAA-increasing agents (e.g., a HPPD inhibitor such as nitisinone) are suitable for administration in conjunction with or in the same pharmaceutical composition as a second therapeutic agent. In certain embodiments, the second therapeutic agent is an agent useful to treat insulin resistance, type 2 diabetes, or metabolic syndrome; or a tyrosine dietary supplement. Such therapeutic agents include a meglitinide, a sulfonurea, a dipeptidyl peptidase 4 inhibitor, a biguanide, a thiazolidinedione, an alpha glucosidase inhibitor, an amylin mimetic, an incretin mimetic, or an appetite suppressant. Examples of these second therapeutic agents include Repaglinide (Prandin®), Nateglinide (Starlix®), Glipizide (Glucotrol®), Glimepiride (Amaryl®), Glyburide (Diabeta®, Glynase®), Saxagliptin (Onglyza®), Sitagliptin (Januvia), Linagliptin (Tradjenta®), Metformin (Fortamet®, Glucophage®), Rosiglitazone (Avandia®), Pioglitazone (Actos®), Acarbose (Precose®), Miglitol (Glyset®), Pramlintide (Symlin®), Exenatide (Byetta®), Liraglutide (Victoza®), Orlistat (Xenical®), Sibutramine (Meridia®), Phendimetrazine tartrate (Bontril®), Methamphetamine, Phentermine (Adipex-P®), Oxyntomodulin, an oxyntomodulin analog, PYY (Peptide YY), PYY analog, GLP-1 and a GLP-1 analog.

In certain embodiments, the second therapeutic agent is useful to treat obesity. Agents useful to treat obesity include lipase inhibitors, intestinal microsomal triglyceride transfer protein (MTP) inhibitors, diacylglycerol O-acyltransferase inhibitors, low-affinity sodium-dependent glucose co-transporters (SGLT2), gastrointestinal G-protein-coupled receptor (GPR119) agonists, endocannabinoid receptor blockers, amylin analogs, PYY analogs, pancreatic peptide analogs, GLP-1 analogs, dopamine and norepinephrine reuptake inhibitors, 5-HT2C agonists, dopaminergic ligands, melanocortin 4 receptor agonists, melanin-concentrating hormone receptor 1 agonists, histamine H3 receptor agonists, neuropeptide Y antagonists, Agouti-related protein inhibitors, protein tyrosine phosphatase 1B inhibitors, 11-B hydroxysteroid dehydrogenase type 1 inhibitors, methionine aminopeptidase-2 inhibitors and brown adipose tissue activators. Examples of the obesity-treating agents include cetilistat, orlistat, GT 389-255, lomitapide, SLX-4090, JNJ-16269110, PF-04620110, GW-869682, JNJ-28431754, GSK-189075, MBX-2982, PSN-821, TM-38837, pramlintide, metreleptin, PYY3-36, TM-30339, exenatide, exenatide LAR, liraglutide, bupropion, Qsymia® (phentermine and topiramate), Contrave® (bupropion and naltrexone), Empatic® (bupropion and zonisamide), lorcaserin, GSK-598809, MK-0493, BMS-830216, HPP-404, SCH-497079, velneperit, MK-0557, TTP-435, trodusquemine (MSI-1436), INCB-13739, ZGN-433, irisin and BMP7.

Mammals treated by the methods described herein include mammals susceptible to or suffering from a disease or condition selected from insulin resistance, type 2 diabetes, or metabolic syndrome. In certain embodiments, the mammal does not suffer from adolescent type I tyrosinemia, but suffers from one or more of the other conditions described herein.

In certain embodiments, a mammal treated or selected for treatment by methods described herein is obese. Some obese mammals exhibit metabolic syndrome (also known as “syndrome X”), which is characterized by a combination of two or more the following (typically three or more): visceral obesity, dyslipidemia (low HDL-cholesterol, raised VLDL-triglycerides), hyperglycemia (raised fasting glucose), insulin resistance, hypertension (raised blood pressure), and microalbuminuria (elevated urinary albumin excretion). Patients exhibiting symptoms of metabolic syndrome/syndrome X are at high risk of developing type 2 diabetes, cardiovascular disease and/or cancers. In particular embodiments, an obese mammal suffers from type II diabetes, cardiovascular disease or cancer.

In certain embodiments, a mammal treated or selected for treatment by methods described herein is suffering from a disease or condition associated with elevated free fatty acid plasma concentrations or incomplete lipid (triglyceride) oxidation. Such diseases or conditions include insulin resistance; diabetes, Metabolic syndrome, atherosclerosis, inflammation associated with insulin resistance, diabetes, Metabolic syndrome, or atherosclerosis (elevated FFAs provoke inflammation in endothelial cells, among peripheral tissues including adipose and muscle and reduction of circulating FFAs has been correlated to reduced inflammatory markers including CRP and inflammatory cytokines; (Santomauro et al, 1999; and Gregorio et al 1997)); cardiovascular disease (Pirro et al, 2002); immunosuppression (Stulnig et al, 2000); non-alcoholic fatty-acid pancreas disease (Mathur et al, 2007); nonalcoholic fatty-liver disease (Ibrahim et al, 2011); muscle myopathy and wasting; genetic disorders of lipid metabolism, such as Wolman's disease, fatty acid oxidation disorders, such as MCAD deficiency, and neutral lipid storage disease; and diabetic and non-diabetic retinopathy.

In certain embodiments, the mammal treated or selected for treatment by methods described herein is a human. In particular embodiments, the human is at risk of developing type II diabetes. For example, the human at risk of developing type II diabetes has (e.g., is determined to have) a plasma concentration of 1, 2, or all 3 BCAAs that is greater than corresponding plasma concentrations of a human not having or at risk of developing type II diabetes. In some embodiments, the human at risk for developing type II diabetes has (e.g., is determined to have) a plasma concentration of one, two or all three BCAAs that is greater than one standard deviation above the mean for a human. In an alternate embodiment, the human at risk of developing type II diabetes has (e.g., is determined to have) a plasma concentration of one, two or all three BCAAs that is in the highest 25% of corresponding plasma concentrations in a population of humans. In some embodiments, the human at risk of developing type II diabetes has (e.g., is determined to have) a plasma BCAA concentration of one or more of the following: greater than 150 μM, 175 μM, 200 μM, 250 μM, 275 μM, or 300 μM valine; greater than 75 μM, 100 μM, 125 μM, 150 μM, or 175 μM leucine; and greater than 40 μM, 50 μM, 60 μM, 70 μM, 80 μM, 90 μM, or 100 μM isoleucine. In a particular embodiment, the human at risk of developing type II diabetes has (e.g., is determined to have) a plasma BCAA concentration greater than or equal to one, two or three of the following plasma BCAA concentrations:
(1) valine: 250, 260 or 270 μM
(2) leucine: 115, 120, 125 or 130 μM
(3) isoleucine: 65, 70 or 75 μM.
   Alternatively, the human at risk of developing type II diabetes has (e.g., is determined to have) a sum total plasma leucine and isoleucine concentration that is greater than or equal to 160, 170 or 180 μM. In exemplary embodiment, the human at risk for developing type II diabetes has (e.g., is determined to have) a plasma concentration greater than 270 μM valine, greater than 130 μM leucine and greater than 75 μM isoleucine.

### Uses, Formulation and Administration

### Pharmaceutically Acceptable Compositions

Agents useful in the methods described herein can be formulated into compositions comprising the agent (optionally in combination with a second therapeutic agent, as described above) and a pharmaceutically acceptable carrier, adjuvant, or vehicle. The amount of the LNAA-increasing agent in compositions of this invention is such that it is effective to measurably increase LNAA concentrations in plasma or intracellularly. In certain embodiments, a composition of this invention is formulated for administration to a mammal in need of such composition. In some embodiments, a composition of this invention is formulated for oral administration to a mammal.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, plasma proteins, such as human plasma albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, Vitamin E polyethylene glycol succinate (d-alpha tocopheryl polyethylene glycol 1000 succinate), sodium carboxymethylcellulose, polyacrylates, waxes, polyethylene-polyoxypropylene-block polymers, and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be an aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of an injectable composition of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Pharmaceutically acceptable compositions of this invention may be orally administered in any orally acceptable dosage form including, but not limited to, capsules, tablets, aqueous and non-aqueous suspensions or solutions. In such solid dosage forms, an LNAA-increasing agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is typically combined with emulsifying and suspending agents. Liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, polyethylene glycol (e.g., PEG 200, PEG 400, PEG 1000, PEG 2000), propylene glycol, 1,3-butylene glycol, dimethylformamide, oils (in particular, cottonseed, groundnut, corn, germ, olive, castor, and sesame oils), glycerol, tetrahydrofurfuryl alcohol, Vitamin E polyethylene glycol succinate (d-alpha tocopheryl polyethylene glycol 1000 succinate), polyethylene glycols and fatty acid esters of sorbitan, and mixtures thereof. If desired, certain sweetening, flavoring or coloring agents may also be added. The liquid forms above can also be filled into a soft or hard capsule to form a solid dosage form. Suitable capsules can be formed from, for example, gelatin, starch and cellulose derivatives (e.g., hydroxycellulose, hydropropylmethylcellulose).

Solid compositions of a similar type may also be employed as fillers in soft and hard-filled capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled capsules using such excipients as lactose or milk sugar as well as high molecular weight polyethylene glycols and the like.

An LNAA-increasing agent can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions of this invention are formulated for oral administration.

The amount of an LNAA-increasing agent that is typically combined with the carrier materials to produce a composition in a single dosage form will vary depending upon the host treated, the particular mode of administration. In certain embodiments, compositions are formulated so that a dosage of between 0.01-100 mg/kg body weight/day of an LNAA-increasing agent can be administered to a patient receiving these compositions. In certain embodiments, the LNAA-increasing agent is nitisinone and composition comprising nitisinone are formulated so that a dosage of between 0.01-0.5 mg/kg body weight/day of an LNAA-increasing agent can be administered to a patient receiving these compositions. In one aspect of these embodiments, nitisinone is formulated so that a dosage of between 0.1-0.5 mg/kg body weight/day of an LNAA-increasing agent can be administered to a patient receiving these compositions. In another aspect of these embodiments, nitisinone is formulated so that a dosage of between 0.01-0.1 mg/kg body weight/day of an LNAA-increasing agent can be administered to a patient receiving these compositions.

In some embodiments, the LNAA-increasing agent is formulated together with a second therapeutic, such as a second therapeutic agent selected from a meglitinide, a sulfonurea, a dipeptidyl peptidase 4 inhibitor, a biguanide, a thiazolidinedione, an alpha glucosidase inhibitor, an amylin mimetic, an incretin mimetic, or an appetite suppressant. In one aspect of these embodiments, the second therapeutic agent is selected from Repaglinide (Prandin®), Nateglinide (Starlix®), Glipizide (Glucotrol®), Glimepiride (Amaryl®), Glyburide (Diabeta®, Glynase®), Saxagliptin (Onglyza®), Sitagliptin (Januvia), Linagliptin (Tradjenta®), Metformin (Fortamet®, Glucophage®), Rosiglitazone (Avandia®), Pioglitazone (Actos®), Acarbose (Precose®), Miglitol (Glyset®), Pramlintide (Symlin®), Exenatide (Byetta®), Liraglutide (Victoza®), Orlistat (Xenical®), Sibutramine (Meridia®), Phendimetrazine tartrate (Bontril®), Methamphetamine, Phentermine (Adipex-P®), Oxyntomodulin, an oxyntomodulin analog, PYY, PYY analog, GLP-1 and a GLP-1 analog.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### Uses of Compounds and Pharmaceutically Acceptable Compositions

LNAA-increasing agents and compositions described herein are generally useful for treating diseases and disorders associated with elevated plasma or intracellular BCAA concentrations, such as insulin resistance, type 2 diabetes and metabolic syndrome. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As discussed above, LNAA-increasing agents are suitable for administration in conjunction with a second therapeutic agent. The additional agents are optionally administered separately from an LNAA-increasing agent-containing composition, as part of a multiple dosage regimen. Alternatively, those agents are part of a single dosage form, mixed together with an LNAA-increasing agent in a single composition. If administered as part of a multiple dosage regime, the two active agents are typically submitted simultaneously, sequentially or within a period of time from one another (e.g., one hour, two hours, six hours, twelve hours, one day, one week, two weeks, one month).

As used herein, the terms “combination,” “combined,” and related terms refer to the simultaneous or sequential administration of therapeutic agents in accordance with this invention. For example, an LNAA-increasing agent is administered with another therapeutic agent simultaneously or sequentially in separate unit dosage forms or together in a single unit dosage form. Accordingly, the present invention provides a single unit dosage form comprising an LNAA-increasing agent, a second therapeutic agent, and a pharmaceutically acceptable carrier, adjuvant, or vehicle.

The amount of an LNAA-increasing agent and a second therapeutic agent (in those compositions which comprise a second therapeutic agent as described above) that is combined with the carrier materials to produce a single dosage form will typically vary depending upon the host treated and the particular mode of administration. Preferably, compositions of this invention are formulated so that a dosage of between 0.01-100 mg/kg body weight/day of an LNAA-increasing agent is administered.

In those compositions that include a second therapeutic agent, that second therapeutic agent and the LNAA-increasing agent may act synergistically. Therefore, the amount of second therapeutic agent in such compositions may be less than that required in a monotherapy utilizing only that therapeutic agent. In such compositions, a dosage of between 0.01 μg/kg body weight/day-1,000 mg/kg body weight/day of the second therapeutic agent is typically administered. The dosage of the second therapeutic will, of course, depend upon the nature of that therapeutic and its recommended dosages in a monotherapy or other uses.

The amount of second therapeutic agent present in the compositions of this invention will typically be no more than the amount that would normally be administered in a composition comprising that therapeutic agent as the only active agent. Preferably, the amount of second therapeutic agent in the presently disclosed compositions will range from 50% to 100% of the amount normally present in a composition comprising that agent as the only therapeutically active agent.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

### Example 1

The dosing solutions were prepared the day of study dosing, approximately one hour prior to dosing. Compound 1 is nitisinone (also referred to herein as “NTBC”).

54 C56BL/6 mice were housed for at least 3 days prior to dosing and were fully acclimated at study start. All mice were ad libitum fed throughout the study. Water was given ad libitum throughout the holding and study periods. Body weight was measured prior to dosing. Dosing for Group 2 and 3 occurred once on the day of study.

TABLE A
Overall Design

| | | | | | Dose |
|---|---|---|---|---|---|
| Group | | | | Number of | (active |
| ID | Compound | Formulation | Route | Animals | basis) |
| 1 | Naive | N/A | NA | 6 | NA |
| 2 | Vehicle | aqueous | Oral Gavage | 12 | 0 mg/kg |
| | | | (PO) | | |
| 3 | Compound 1 | aqueous | Oral Gavage | 12 | 1 mg/kg |
| | | | (PO) | | |

TABLE B
SAMPLE COLLECTION

| | | Collection |
|---|---|---|
| Data Type | Data Points | Times |
| Plasma and Tissue | 1 time point pre-dosing (n = 6) | Starting |
| Collection | or 2 time points (n = 6) in 12 | at 7:00 AM |
| | hrs of a dose administration | |
| | (see Table below for time | |
| | points per group) | |
| Body Weight | Once on day of dosing | 7:00 AM |

Collection Points
Time Post Dosing
Hours

| | 0 | 1 | 2 | 4 | 6 | 8 | 10 | 12 |
|---|---|---|---|---|---|---|---|---|
| Group 1 | ✓ | | | | | | | |
| Groups 2 | | | | | ✓ | | | ✓ |
| and 3 | | | | | | | | |

Mice had blood collected at sacrifice (by CO₂ asphyxiation) by cardiac puncture at the collection time points shown in the table above. Blood collection (approximately 600 μL) was into pre-chilled (0-4° C.) K3-EDTA containing polypropylene blood collection tubes. After blood collection, blood samples were maintained chilled (2-6° C.) and centrifuged within 30 minutes. The collected plasma (approximately 300 μL) was placed in sample tubes and immediately stored at nominally −80° C. Frozen plasma samples were shipped for analysis on dry ice.

Samples were extracted and split into equal parts for analysis on GC/MS and LC/MS/MS platforms. Proprietary software was used to match ions to a library of standards for metabolite identification and for metabolite quantitation by peak area integration.

Six hours following dosing with Compound 1, the treated mice had reduced plasma levels of the BCAAs valine, isoleucine and leucine (FIG. 2A). At the same time point, plasma levels of tyrosine and tyrosine metabolites increased above control levels (FIG. 2B).

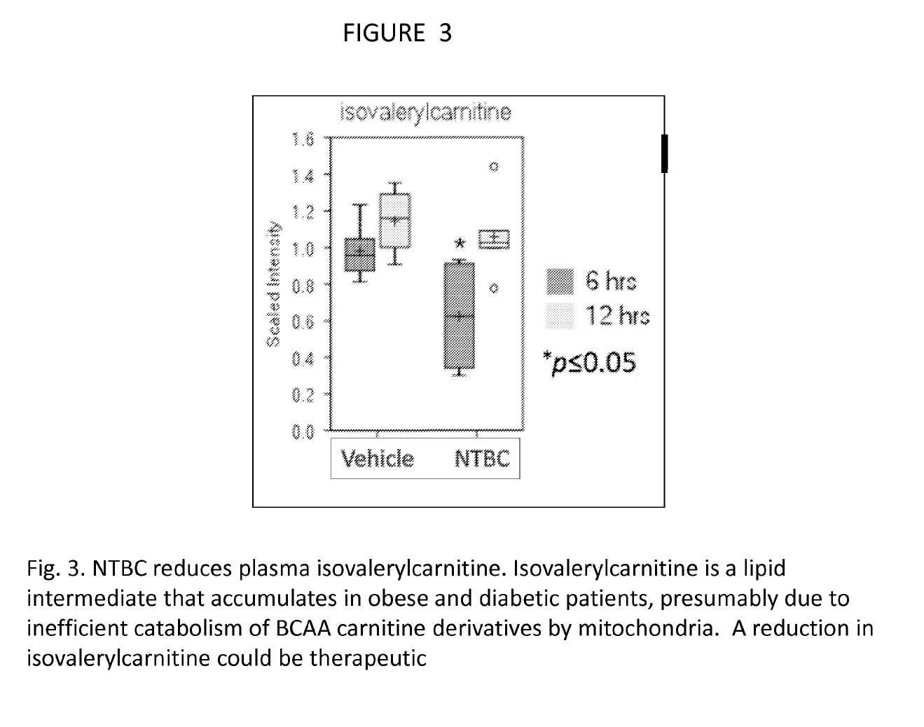

We then measured plasma levels of isovalerylcarnitine. Isovalerylcarnitine is a lipid intermediate that accumulates in obese and diabetic patients, presumably due to inefficient catabolism of BCAA carnitine derivatives by the mitochondria. After six hours there was a statistically significant decrease in isovalerylcarnitine levels as compared to vehicle alone (FIG. 3). Reducing C3 and C5 acylcarnitines in obese animals is associated with health benefits. Isovalerylcarnitine levels were higher in the muscles of obese rats as compared to lean rats and were reduced by exercise, together with a restoration of insulin sensitivity and glucose tolerance (Chavez et al. 2003).

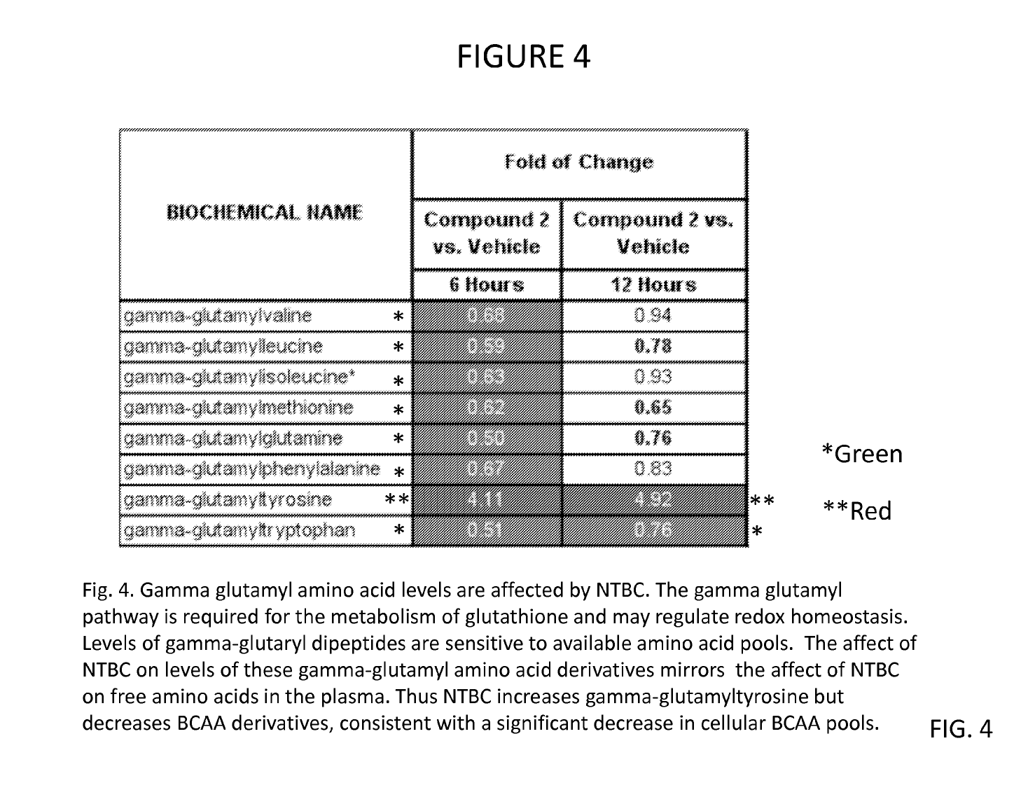

Next we measured plasma levels of various gamma glutamyl amino acids. The gamma glutamyl pathway is required for the metabolism of glutathione and may regulate redox homeostasis. Levels of gamma-glutaryl dipeptides are sensitive to available amino acid pools. The affect of NTBC on levels of these gamma-glutamyl amino acid derivatives mirrors the affect of NTBC on free amino acids in the plasma (cf. FIG. 4 and FIG. 1). As shown in FIG. 4, six hours post-dosing, NTBC causes a statistically significant increase in gamma-glutamyltyrosine, and a statistically significant decrease in BCAA derived gamma-glutaryl amino acids. This result is consistent with NTBC reducing intracellular pools of BCAAs.

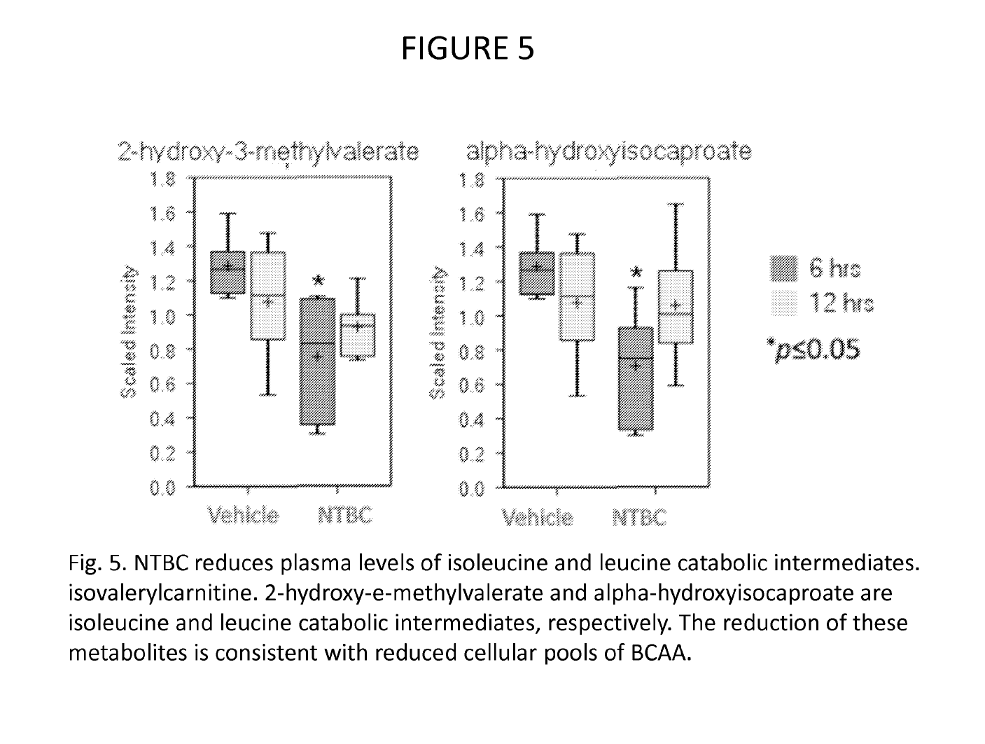

We then measured plasma levels of 2-hydroxy-3-methylvalerate and α-hydroxyisocaproate. These are metabolites arising from the catabolism of isoleucine and leucine, respectively. As shown in FIG. 5, NTBC caused a statistically significant decrease in both 2-hydroxy-3-methylvalerate and α-hydroxyisocaproate six hours post-dosing. This result is also consistent with reduced cellular pools of BCAAs, specifically isoleucine and leucine.

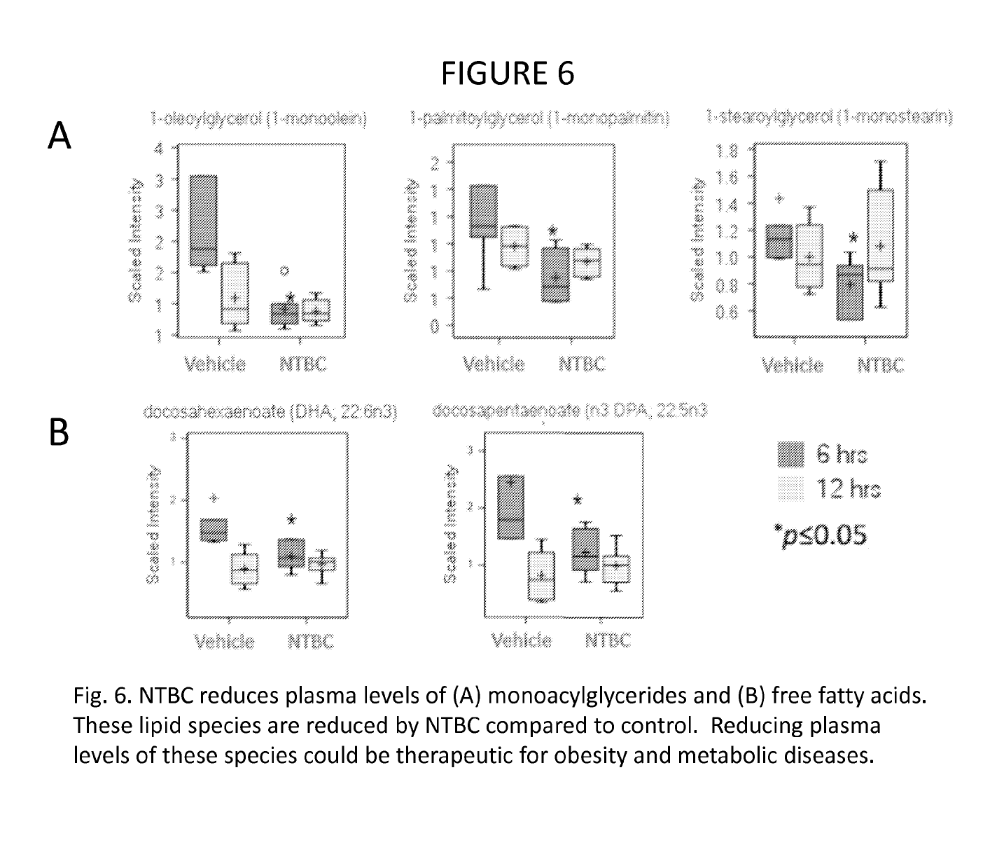

The accumulation of free (nonesterified) fatty acids in plasma of rodents and humans mediates insulin resistance and other symptoms of metabolic disease. Therefore, we also looked at levels of various lipid species in the NTBC treated mice. FIG. 6 demonstrates that six hours post-dosing NTBC caused statistically significant reductions in triacylglyceride breakdown intermediates such as monoacylglycerols 1-oleoglycerol, 1-palmitoylglycerol, and 1-stearoylglycerol (Panel A), as well as in the free fatty acids docosahexaenoate and docosapentaenoate (Panel B) as compared to vehicle alone. These results support the conclusion that the inhibition of HPPD by NTBC causes an accumulation of tyrosine, which causes a reduction in BCAA levels, which directly or indirectly, increases complete oxidation of FFA as evident by the decreased fatty acid species in plasma.

The lack of a statistically significant reduction in BCAAs, isovalerylcarnitine, gamma glutamyl amino acids, 2-hydroxy-3-methylvalerate, α-hydroxyisocaproate, monoacylglycerols and free fatty acids at 12 hr post treatment is presently unexplainable, but could be due to the fact that the mice were lean, healthy animals so the level of these metabolites were not elevated to begin with, as opposed to what would be expected from obese or diabetic animals. The results presented provide a proof of concept that increasing LNAAs (and in particular tyrosine through NTBC treatment) can cause a reduction in BCAAs, their metabolites and catabolites, and incompletely oxidized lipid molecules, all known to be elevated in obese, diabetic and metabolic disease patients.

### REFERENCES

1. Beaudegnies, R. et al. (2009) Bioorganic Med. Chem. 17, 4134-4152.
2. Felig, P. et al. (1969) N. Engl. J. Med. 281, 811-816.
3. Lock. E. A. et al. (1996) Tox. App. Pharm. 141, 439-447.
4. Lock, E. A. et al. (1998) J. Inher. Metab. Dis. 21, 498-506.
5. Lock, E. A. et al. (2000) Toxicology 144, 179-187.
6. Newgard, C. B. (2009) Cell Metab. 9, 311-326.
7. Newgard, C. B. (2012) Cell Metab. 15, 606-614.
8. Van Spronsen, F. J. et al. (2001) Am J. Clin Nutr. 73, 153-157.
9. Van Spronse, F. J. et al. (2010) J. Inherit. Metab. Dis. 33, 671-676.
10. Verrey, F. (2003) Pflugers Arch. Eur. J. Physiol. 445, 529-533.
11. Wang et al. (2011) Nature Med. 17, 448-454.
12. Xiao, F. et al. (2011) Diabetes 60, 746-756.
13. Zhang, V. et al. (2007) Diabetes 56, 1647-1654.
14. Muoio, D. M. et al. (2008) Nat. Rev. Mol. Cell Biol. 9, 193-205.
15. Chavez, J. A. et al. (2003) Arch. Biochem. Biophys. 419, 101-109.
16. Pietz, J. et al. (1999) J. Clin. Invest. 103, 1169-1178.
17. Efron, M. L. et al. (1969) J. Pediatrics, 74, 399-405.
18. Strauss, K. A. et al. (2010) Mol. Gen. Metab. 99, 333-345.
19. Santomauro, A. T. M. G. et al (1999) Diabetes, 48,
20. Gregorio, F. et al. (1997) Diabetes Res. Clin. Pract., 37, 21-33.
21. Pirro, Metal. (2002) Atherosclerosis, 160, 377-384.
22. Stulnig, T. M. et al. (2000) FASEB J, 14:939-947.
23. Ibrahim, S. H. et al. (2011) J Pediatr Gastroenterol Nutr. 53, 131-140.
24. Mathur, A. et al. (2007) HPB (Oxford), 9:312-318.

1. A composition for reducing the plasma concentration of at least one branched chain amino acid in a mammal comprising an agent that increases the plasma concentration of at least one, but not all, large neutral amino acids, wherein the agent is not a leucine supplement.

2. A composition for reducing the plasma concentration of at least one free fatty acid and/or at least one fatty acid oxidation metabolite, and/or at least one metabolite of triglyceride oxidation in a mammal comprising an agent that increases the plasma concentration of at least one, but not all, large neutral amino acids, wherein the agent is not a leucine supplement.

3. The composition of claim 2 for reducing the plasma concentration of at least one free fatty acid or at least one monoacylglycerides.

4. The composition of claim 1, wherein the agent increases the plasma concentration of one or more of tyrosine, phenylalanine, tryptophan, methionine, or histidine.

5. The composition of claim 1, wherein the agent is other than an ingestible source of amino acids.

6. The composition of claim 1, wherein the agent increases plasma concentration of tyrosine.

7. The composition of claim 6, wherein the agent is a hydroxyphenylpyruvate dioxygenase (HPPD) inhibitor.

8. The composition of claim 7, wherein the HPPD inhibitor is nitisinone.

9. The composition of claim 1, for treating or preventing a disease or condition selected from insulin resistance; diabetes; Metabolic syndrome; atherosclerosis; inflammation associated with insulin resistance, diabetes, Metabolic syndrome, or atherosclerosis (elevated FFAs provoke inflammation in endothelial cells, among peripheral tissues including adipose and muscle and reduction of circulating FFAs has been correlated to reduced inflammatory markers including CRP and inflammatory cytokines); cardiovascular disease; immunosuppression; non-alcoholic fatty-acid pancreas disease; nonalcoholic fatty-liver disease; muscle myopathy and wasting; genetic disorders of lipid metabolism, such as Wolman's disease, fatty acid oxidation disorders, such as MCAD deficiency, and neutral lipid storage disease; and diabetic and non-diabetic retinopathy.

10. The composition of claim 9, for treating or preventing a disease or condition selected from insulin resistance, type 2 diabetes, and metabolic syndrome.

11. The composition of claim 10, for use in combination with a second therapeutic agent selected from an agent useful to treat insulin resistance, type 2 diabetes, or metabolic syndrome; or a tyrosine dietary supplement.

12. The composition of claim 11, for use in combination with a second therapeutic agent selected from a meglitinide, a sulfonurea, a dipeptidyl peptidase 4 inhibitor, a biguanide, a thiazolidinedione, an alpha glucosidase inhibitor, an amylin mimetic, an incretin mimetic, or an appetite suppressant.

13. The composition of claim 12, for use in combination with a second therapeutic agent selected from Repaglinide (Prandin®), Nateglinide (Starlix®), Glipizide (Glucotrol®), Glimepiride (Amaryl®), Glyburide (Diabeta®, Glynase®), Saxagliptin (Onglyza®), Sitagliptin (Januvia), Linagliptin (Tradjenta®), Metformin (Fortamet®, Glucophage®), Rosiglitazone (Avandia®, Pioglitazone (Actos®), Acarbose (Precose®), Miglitol (Glyset®), Pramlintide (Symlin®), Exenatide (Byetta®), Liraglutide (Victoza®), Orlistat (Xenical®), Sibutramine (Meridia®), Phendimetrazine tartrate (Bontril®, Methamphetamine, Phentermine (Adipex-P®), Oxyntomodulin, an oxyntomodulin analog, PYY, PYY analog, GLP-1 and a GLP-1 analog.

14. The composition of claim 1, for the treatment of an obese mammal.

15. The composition of claim 14, for use in combination with a second therapeutic agent useful to treat obesity.

16. The composition of claim 1, for the treatment of a human.

17. The composition of claim 16, for the treatment of a human determined to have a plasma BCAA concentration of any one or more of; greater than 250 μM valine, greater than 115 μM leucine, greater than 65 μM isoleucine prior to treatment.

18. A method of reducing the plasma concentration of at least one branched chain amino acid in a mammal in need of such reduction comprising the step of administering to the mammal an agent that increase the plasma concentration of at least one, but not all, large neutral amino acids, wherein the agent is not a leucine supplement.

19. A method of reducing the plasma concentration of at least one free fatty acid and/or at least one fatty acid oxidation metabolite, and/or at least one metabolite of triglyceride oxidation in a mammal in need of such reduction comprising the step of administering to the mammal an agent that increases the plasma concentration of at least one, but not all, large neutral amino acids, wherein the agent is not a leucine supplement.

20. The method of claim 19, wherein the plasma concentration of at least one free fatty acid or at least one monoacylglyceride is reduced.

21. The method of claim 18, wherein the agent increases the plasma concentration of one or more of the tyrosine, phenylalanine, tryptophan, methionine, or histidine.

22. The method of claim 18, wherein the agent is other than an ingestible source of amino acids.

23. The method of claim 21, wherein the agent increases plasma concentration of tyrosine.

24. The method of claim 23, wherein the agent is a hydroxyphenylpyruvate dioxygenase (HPPD) inhibitor.

25. The method of claim 24, wherein the HPPD inhibitor is nitisinone.

26. The method of claim 18, wherein the mammal is susceptible to or suffering from a disease or condition selected from insulin resistance; diabetes; Metabolic syndrome; atherosclerosis; inflammation associated with insulin resistance, diabetes, Metabolic syndrome, or atherosclerosis (elevated FFAs provoke inflammation in endothelial cells, among peripheral tissues including adipose and muscle and reduction of circulating FFAs has been correlated to reduced inflammatory markers including CRP and inflammatory cytokines); cardiovascular disease; immunosuppression; non-alcoholic fatty-acid pancreas disease; nonalcoholic fatty-liver disease; muscle myopathy and wasting; genetic disorders of lipid metabolism, such as Wolman's disease, fatty acid oxidation disorders, such as MCAD deficiency, and neutral lipid storage disease; and diabetic and non-diabetic retinopathy.

27. The method of claim 26, wherein the mammal is susceptible to or suffering from a disease or condition selected from insulin resistance; type 2 diabetes, or metabolic syndrome.

28. The method of claim 27, comprising the additional step of co-administering to the mammal a second therapeutic agent selected from an agent useful to treat insulin resistance, type 2 diabetes, or metabolic syndrome; or a tyrosine dietary supplement.

29. The method of claim 28, wherein the second therapeutic agent is selected from a meglitinide, a sulfonurea, a dipeptidyl peptidase 4 inhibitor, a biguanide, a thiazolidinedione; an alpha glucosidase inhibitor, an amylin mimetic, an incretin mimetic, or an appetite suppressant.

30. A method for claim 29, wherein the second therapeutic agent selected from Repaglinide (Prandin®), Nateglinide (Starlix®), Glipizide (Glucotrol®), Glimepiride (Amaryl®), Glyburide (Diabeta®, Glynase®), Saxagliptin (Onglyza®), Sitagliptin (Januvia), Linagliptin (Tradjenta®), Metformin (Fortamet®, Glucophage®), Rosiglitazone (Avandia®, Pioglitazone (Actos®), Acarbose (Precose®), Miglitol (Glyset®), Pramlintide (Symlin®), Exenatide (Byetta®), Liraglutide (Victoza®), Orlistat (Xenical®), Sibutramine (Meridia®), Phendimetrazine tartrate (Bontril®, Methamphetamine, Phentermine (Adipex-P®), Oxyntomodulin, an oxyntomodulin analog, PYY, PYY analog, GLP-1 and a GLP-1 analog.

31. The method of claim 18, wherein the mammal is obese.

32. The method of claim 32, comprising the additional step of co-administering to the mammal a second therapeutic agent useful to treat obesity.

33. The method of claim 18, wherein the mammal is a human.

34. The method of claim 33, wherein the human has been determined to have a plasma BCAA concentration of any one or more of; greater than 250 μM valine, greater than 115 μM leucine, and/or greater than 65 μM isoleucine prior to said administration of the agent.

35. A pharmaceutical composition comprising:
a. a HPPD inhibitor;
b. a second therapeutic agent selected from an agent useful to treat insulin resistance, type 2 diabetes, metabolic syndrome, or obesity; or a dietary tyrosine supplement; and
c. a pharmaceutically acceptable carrier.

36. The pharmaceutical composition of claim 35, wherein the HPPD inhibitor is nitisinone.

37. The pharmaceutical composition of claim 35, wherein the second therapeutic agent is selected from a meglitinide, a sulfonurea, a dipeptidyl peptidase 4 inhibitor, a biguanide, a thiazolidinedione; an alpha glucosidase inhibitor, an amylin mimetic, an incretin mimetic, or an appetite suppressant.

38. The pharmaceutical composition of claim 37, wherein the second therapeutic agent selected from Repaglinide (Prandin®), Nateglinide (Starlix®), Glipizide (Glucotrol®), Glimepiride (Amaryl®), Glyburide (Diabeta®, Glynase®), Saxagliptin (Onglyza®), Sitagliptin (Januvia), Linagliptin (Tradjenta®), Metformin (Fortamet®, Glucophage®), Rosiglitazone (Avandia®, Pioglitazone (Actos®), Acarbose (Precose®), Miglitol (Glyset®), Pramlintide (Symlin®), Exenatide (Byetta®), Liraglutide (Victoza®), Orlistat (Xenical®), Sibutramine (Meridia®), Phendimetrazine tartrate (Bontril®, Methamphetamine, Phentermine (Adipex-P®), Oxyntomodulin, an oxyntomodulin analog, PYY, PYY analog, GLP-1 and a GLP-1 analog.
